SEQUENCE LISTING (11 sequences)

SEQ ID NO: 1 - protein, 348 aa, Oryza sativa
MGDEKAAPKAGATADPVVLRMELHCAGCAQKVKKSIKHLAGVESVAADVATNTVVVAGTAEAAALKARIEAKTKKPVEVVSAGGGGAAAKKPAAEPKAVKDDGGEKKDAQAKEEKGKKQPPEEKKPKEETVLLRIRLHCDGCADRIRRRIYKIKGVKEVVMDGNAKDEVKVSGTMDVPAMLTYLTEKLNRAVEAVAPGSKKDEKKKDKGGDADGGEKKKDAAGGDKKDKGKSIEVAGPSTAAAAASMAPAPAEASTYHVSPYGHGYFAYPQQQGPPPSYYQYYGGGNGDGVGYANPNAGGAGGYYHPHPNDVPTYQPPPSYPPYPYQLDMSPAPQLFSDENPNACSVM

SEQ ID NO: 2 - DNA, 1047 bp, Oryza sativa
  source 1..1047: mol_type = genomic DNA
atgggggatgagaaggctgctccgaaggcgggcgccaccgccgacccggtggtgctgaggatggagttgcattgcgccggctgcgcgcagaaggtcaagaagtccatcaagcacttggccggggtggagtcggtggcggcggacgtggcgacgaacacggtcgtcgtggcggggacggccgaagcggccgcgctcaaggcgaggatcgaggcgaagaccaagaagcccgtcgaggttgtctccgccggcggcggtggcgccgccgccaagaaacccgccgccgagcccaaggccgtcaaggacgatggcggcgagaagaaggacgcccaggcgaaagaggagaaggggaagaagcaaccgccagaggagaagaaacccaaagaggaaacggtgctgctcaggattcgcctccactgcgatggctgcgccgaccgcatcaggcgacgcatctacaagatcaaaggggtgaaggaagtggtaatggacggcaatgccaaggatgaggtgaaggtgtcgggcacaatggacgtgccggccatgctgacctatctcaccgagaagctcaaccgcgccgtggaggccgtggcgcccggcagcaagaaagacgagaagaagaaggacaagggcggtgacgccgacggcggcgagaagaagaaagacgccgccggcggcgacaagaaggacaaaggcaagagcatcgaggtcgccggcccgtccaccgccgccgccgcggcgtccatggcgccggcgccggccgaggcgagcacgtaccacgtctcgccgtacggccacggctacttcgcgtacccgcagcagcagggacctccccccagctactaccagtactacggcggcggcaatggcgacggcgtgggctacgccaaccccaatgccggcggcgccggcggctactaccacccccaccccaacgacgtccctacctaccagccgccgccgtcgtacccgccgtacccctaccagctcgacatgtcgccggcgccgcagctgttcagcgacgagaaccccaacgcctgctcggtgatgtga

SEQ ID NO: 3 - DNA, 20 bp, synthetic construct
  source 1..20: mol_type = other DNA
cgcgctcaaggcgaggatcg

SEQ ID NO: 4 - DNA, 24 bp, synthetic construct
  source 1..24: mol_type = other DNA
ggcacgcgctcaaggcgaggatcg

SEQ ID NO: 5 - DNA, 24 bp, synthetic construct
  source 1..24: mol_type = other DNA
aaaccgatcctcgccttgagcgcg

SEQ ID NO: 6 - DNA, 22 bp, synthetic construct
  source 1..22: mol_type = other DNA
ctgccgcctctcgtttctttct

SEQ ID NO: 7 - DNA, 22 bp, synthetic construct
  source 1..22: mol_type = other DNA
gcgttggtagggttgcctcttg

SEQ ID NO: 8 - DNA, 20 bp, synthetic construct
  source 1..20: mol_type = other DNA
atgggggatgagaaggctgc

SEQ ID NO: 9 - DNA, 20 bp, synthetic construct
  source 1..20: mol_type = other DNA
tcacatcaccgagcaggcgt

SEQ ID NO: 10 - DNA, 19 bp, synthetic construct
  source 1..19: mol_type = other DNA
cgcgctcaaggcgaggtcg

SEQ ID NO: 11 - DNA, 21 bp, synthetic construct
  source 1..21: mol_type = other DNA
cgcgctcaaggcgaggagtcg

(19) United States
(12) Patent Application Publication
(10) Pub. No.: US 2026/0176648 A1
(43) Pub. Date: Jun. 25, 2026

(54) USE OF OsHIPP36 GENE IN REGULATING ARSENIC TOLERANCE IN RICE

(71) Applicant: Institute of Soil Science, CAS, Nanjing City (CN)

(72) Inventors: Renfang SHEN, Nanjing City (CN); Xiaofang ZHU, Nanjing City (CN); Lin WANG, Nanjing City (CN); Lu ZHENG, Nanjing City (CN)

(73) Assignee: Institute of Soil Science, CAS, Nanjing City (CN)

(21) Appl. No.: 19/429,451

(22) Filed: Dec. 22, 2025

(30) Foreign Application Priority Data
Dec. 24, 2024 (CN) 202411907272.X

(51) Int. Cl. C12N 15/82; C12N 9/22; C12N 15/11

(52) U.S. Cl. CPC C12N 15/8274; C12N 9/226; C12N 15/11; C12N 15/8202; C12N 2310/20

(57) ABSTRACT

Use of OsHIPP36 gene in the regulation of arsenic tolerance in rice is provided, which belongs to the field of genetic engineering. The present disclosure provides the use of OsHIPP36 gene in the regulation of arsenic tolerance in rice, where protein encoded by the OsHIPP36 gene has the amino acid sequence of SEQ ID NO: 1. The present disclosure obtains a oshipp36 mutant plant by constructing a transgenic vector. After treatment with the external addition of As(III), the mutant plant is found to have significantly increased tolerance to As(III). In particular, the oshipp36 mutant plant after As(III) treatment has significantly longer roots than a Nipponbare wild-type plant, suggesting that the OsHIPP36 gene plays an important role in regulating arsenic tolerance in rice.

### CROSS REFERENCE TO RELATED APPLICATION

This patent application claims the benefit and priority of Chinese Patent Application No. 202411907272.X filed with the China National Intellectual Property Administration on Dec. 24, 2024, the disclosure of which is incorporated by reference herein in its entirety as part of the present application.

### REFERENCE TO SEQUENCE LISTING

A computer readable XML file entitled “SEQUENCE LISTING”, that was created on Jan. 29, 2026, with a file size of about 11899 bytes, contains the sequence listing for this application, has been filed with this application, and is hereby incorporated by reference in its entirety.

### TECHNICAL FIELD

The present disclosure belongs to the field of genetic engineering and, in particular, relates to the use of OsHIPP36 gene in the regulation of arsenic tolerance in rice.

### BACKGROUND

Arsenic is a toxic metalloid that is prevalent in the environment, primarily originating from mining and smelting, fossil fuel combustion, and agricultural activities, etc. Extensive arsenic contamination of the soil poses an even greater threat to the growth of rice. Rice soil in flooded condition provides a anaerobic environment, where arsenic is more easily reduced from pentavalent to trivalent arsenic, and As(III) is more soluble in water with increased mobility and root adsorption. As(III) is mainly absorbed by the rice roots via the silicon absorption pathway, and accordingly, as a silicon superaccumulating plant, rice is more likely to absorb As(III) from the environment. As(V) may also be taken up by rice via the phosphorus transport pathway.

In organisms, the movement of metal ions is usually accomplished by means of metalloproteins. Heavy metal-associated isoprenylated plant proteins (HIPPs) constitute a large family of metalloproteins whose specific structure enables them to bind metal ions and serve as intracellular carriers for metal ion transport, and thus, HIPPs have also been shown to play a role in the accumulation and detoxification of heavy metal ions. At present, whether rice HIPP genes affect arsenic tolerance in rice has rarely been reported.

### SUMMARY

An object of the present disclosure is to provide the use of OsHIPP36 gene in the regulation of arsenic tolerance in rice, which has potential application value for improving arsenic tolerance in rice.

The present disclosure provides the use of OsHIPP36 gene in the regulation of arsenic tolerance in rice, where protein encoded by the OsHIPP36 gene has the amino acid sequence of SEQ ID NO: 1.

Preferably, a CDS sequence of the OsHIPP36 gene has the nucleotide sequence of SEQ ID NO: 2.

Preferably, the regulation of arsenic tolerance in rice includes the step of knocking out or knocking down the OsHIPP36 gene to increase arsenic tolerance in rice.

The present disclosure also provides a sgRNA targeting OsHIPP36 gene, having the nucleotide sequence of SEQ ID NO: 3.

Preferably, the sgRNA includes a sgRNA-S having the nucleotide sequence of SEQ ID NO: 4 and a sgRNA-A having the nucleotide sequence of SEQ ID NO: 5.

The present disclosure also provides a recombinant vector for editing OsHIPP36 gene, the recombinant vector including the sgRNA as described in the above scheme.

The present disclosure also provides a method for increasing arsenic tolerance in rice by utilizing a CRISPR/Cas9 system, including the step of:
introducing the recombinant vector as described in the above scheme into a recipient rice to edit OsHIPP36 gene.

The present disclosure also provides a mutant of OsHIPP36 gene related to arsenic tolerance in rice, where the mutant of the OsHIPP36 gene includes a oshipp36-1 mutant and/or a oshipp36-3 mutant; the oshipp36-1 mutant has a deletion of an A base at position 205 bp within the OsHIPP36 gene fragment having the nucleotide sequence of SEQ ID NO: 2; the oshipp36-3 mutant has a G base inserted between positions 205 bp and 206 bp within the OsHIPP36 gene fragment having the nucleotide sequence of SEQ ID NO: 2.

The present disclosure also provides the use of the mutant of the OsHIPP36 gene as described in the above scheme in the regulation of arsenic tolerance in rice.

The present disclosure also provides a set of primers for identifying the mutant of the OsHIPP36 gene as described in the above scheme, including an upstream primer having the nucleotide sequence of SEQ ID NO: 6 and a downstream primer having the nucleotide sequence of SEQ ID NO: 7.

The present disclosure provides the use of OsHIPP36 gene in the regulation of arsenic tolerance in rice, where protein encoded by the OsHIPP36 gene has the amino acid sequence of SEQ ID NO: 1. The present disclosure constructs a recombinant expression vector for the mutant of the OsHIPP36 gene using CRISPR/Cas9 technology, thereby obtaining homozygous rice lines with mutations in the OsHIPP36 gene. After treatment with the external addition of As(III), the mutant plant is found to have significantly increased tolerance to As(III). In particular, the oshipp36 mutant plant after As(III) treatment has significantly longer roots than a Nipponbare wild-type plant, suggesting that the loss of the OsHIPP36 gene significantly enhances arsenic tolerance in rice, and the OsHIPP36 gene plays an important role in regulating arsenic tolerance in rice. Therefore, the OsHIPP36 gene has potential application value for improving arsenic tolerance in rice. Meanwhile, the present disclosure also lays a theoretical and applied foundation for utilizing the OsHIPP36 gene in breeding arsenic-tolerant rice varieties. The present disclosure obtains a oshipp36 mutant plant by constructing a transgenic vector.

### BRIEF DESCRIPTION OF THE DRAWINGS

In order to illustrate the technical solutions in examples of the present disclosure or in the prior art more clearly, a brief introduction to the accompanying drawings required for the examples will be provided below. Obviously, the accompanying drawings in the following description merely illustrate some of the examples of the present disclosure, and those of ordinary skill in the art may also obtain other drawings according to these drawings without involving any inventive effort.

FIG. 1 shows the results of the identification of oshipp36 mutant plants by CRISPR/Cas9 technology: nip (wild-type Nipponbare): SEQ ID NO: 3; oshipp36-1: SEQ ID NO: 10; oshipp36-3: SEQ ID NO: 11;

FIG. 2 shows the results of a phylogenetic tree analysis of the OsHIPP36 gene;

FIG. 3 shows phenotypic results of wild-type Nipponbare, hipp36-1, and hipp36-3 provided in examples of the present disclosure after arsenic treatment;

FIG. 4 shows root length results of wild-type Nipponbare, hipp36-1, and hipp36-3 provided in examples of the present disclosure after arsenic treatment.

### DETAILED DESCRIPTION OF THE EMBODIMENTS

The principles and features of the present disclosure are described below in conjunction with the accompanying drawings and specific embodiments. The examples provided are intended solely to illustrate the present disclosure and are not meant to limit its scope.

Unless otherwise indicated, all technical and scientific terms used herein have the same meanings as commonly understood by one of ordinary skill in the art to which the present disclosure belongs. Although the present disclosure describes only preferred methods and materials, any methods and materials similar or equivalent to those described herein may also be used in the practice or testing of the present disclosure. All reagents not specified in detail in this application are conventional and commercially available; methods not specified in detail herein are conventional experimental methods known from the conventional technology.

The present disclosure provides the use of OsHIPP36 gene in the regulation of arsenic tolerance in rice, where protein encoded by the OsHIPP36 gene has the amino acid sequence of SEQ ID NO: 1, specifically as follows:

MGDEKAAPKAGATADPVVLRMELHCAGCAQKVKKSIKHLAGVESV
AADVATNTVVVAGTAEAAALKARIEAKTKKPVEVVSAGGGGAAAK
KPAAEPKAVKDDGGEKKDAQAKEEKGKKQPPEEKKPKEETVLLRI
RLHCDGCADRIRRRIYKIKGVKEVVMDGNAKDEVKVSGTMDVPAM
LTYLTEKLNRAVEAVAPGSKKDEKKKDKGGDADGGEKKKDAAGGD
KKDKGKSIEVAGPSTAAAAASMAPAPAEASTYHVSPYGHGYFAYP
QQQGPPPSYYQYYGGGNGDGVGYANPNAGGAGGYYHPHPNDVPTY
QPPPSYPPYPYQLDMSPAPQLFSDENPNACSVM.

In an embodiment of the present disclosure, a CDS sequence of the OsHIPP36 gene has the nucleotide sequence of SEQ ID NO: 2, specifically as follows:

ATGGGGGATGAGAAGGCTGCTCCGAAGGCGGGCGCCACCGCCGAC
CCGGTGGTGCTGAGGATGGAGTTGCATTGCGCCGGCTGCGCGCAG
AAGGTCAAGAAGTCCATCAAGCACTTGGCCGGGGTGGAGTCGGTG
GCGGCGGACGTGGCGACGAACACGGTCGTCGTGGCGGGGACGGCC
GAAGCGGCCGCGCTCAAGGCGAGGATCGAGGCGAAGACCAAGAAG
CCCGTCGAGGTTGTCTCCGCCGGCGGCGGTGGCGCCGCCGCCAAG
AAACCCGCCGCCGAGCCCAAGGCCGTCAAGGACGATGGCGGCGAG
AAGAAGGACGCCCAGGCGAAAGAGGAGAAGGGGAAGAAGCAACCG
CCAGAGGAGAAGAAACCCAAAGAGGAAACGGTGCTGCTCAGGATT
CGCCTCCACTGCGATGGCTGCGCCGACCGCATCAGGCGACGCATC
TACAAGATCAAAGGGGTGAAGGAAGTGGTAATGGACGGCAATGCC
AAGGATGAGGTGAAGGTGTCGGGCACAATGGACGTGCCGGCCATG
CTGACCTATCTCACCGAGAAGCTCAACCGCGCCGTGGAGGCCGTG
GCGCCCGGCAGCAAGAAAGACGAGAAGAAGAAGGACAAGGGCGGT
GACGCCGACGGCGGCGAGAAGAAGAAAGACGCCGCCGGCGGCGAC
AAGAAGGACAAAGGCAAGAGCATCGAGGTCGCCGGCCCGTCCACC
GCCGCCGCCGCGGCGTCCATGGCGCCGGCGCCGGCCGAGGCGAGC
ACGTACCACGTCTCGCCGTACGGCCACGGCTACTTCGCGTACCCG
CAGCAGCAGGGACCTCCCCCCAGCTACTACCAGTACTACGGCGGC
GGCAATGGCGACGGCGTGGGCTACGCCAACCCCAATGCCGGCGGC
GCCGGCGGCTACTACCACCCCCACCCCAACGACGTCCCTACCTAC
CAGCCGCCGCCGTCGTACCCGCCGTACCCCTACCAGCTCGACATG
TCGCCGGCGCCGCAGCTGTTCAGCGACGAGAACCCCAACGCCTGC
TCGGTGATGTGA.

In an embodiment of the present disclosure, a CDS sequence of the OsHIPP36 gene is amplified using the total cDNA of Nipponbare as a template. The OsHIPP36 gene contains 4 exons and 3 introns. The OsHIPP36 gene's cDNA sequence consists of 1047 nucleotides which encode 348 amino acids in total. The OsHIPP36 gene belongs to the HIPP family and has 2 HMA domains.

In an embodiment of the present disclosure, the regulation of arsenic tolerance in rice includes the step of knocking out or knocking down the OsHIPP36 gene to increase arsenic tolerance in rice.

The present disclosure also provides a sgRNA targeting OsHIPP36 gene, having the nucleotide sequence of SEQ ID NO: 3, specifically as follows: 5′-CGCGCTCAAGGCGAGGATCG-3′.

In an embodiment of the present disclosure, the sgRNA includes a sgRNA-S having the nucleotide sequence of SEQ ID NO: 4 and a sgRNA-A having the nucleotide sequence of SEQ ID NO: 5, where the sgRNA-S having the nucleotide sequence of SEQ ID NO: 4 is specifically as follows: 5′-ggcaCGCGCTCAAGGCGAGGATCG-3′; the sgRNA-A having the nucleotide sequence of SEQ ID NO: 5 is specifically as follows:

5′-aaacCGATCCTCGCCTTGAGCGCG-3′.

In an embodiment of the present disclosure, the method for using the sgRNA-S and sgRNA-A includes: mixing the sgRNA-S, sgRNA-A, and ddH₂O, conducting a reaction, cooling the mixture, and obtaining an oligo dimer, where the volume ratio of the sgRNA-S, sgRNA-A and ddH₂O is 5:5:15; the procedure for the reaction is as follows: incubation in a metal bath at 95° C. for 3 min; the procedure of the cooling is as follows: cooling naturally at room temperature for 10 min.

The present disclosure also provides a recombinant vector for editing OsHIPP36 gene, where the recombinant vector includes the sgRNA as described in the above scheme.

In an embodiments of the present disclosure, the backbone vectors for the recombinant vector include SK-gRNA and pC1300-Cas9. In an embodiments of the present disclosure, both the pC1300-Cas9 and SK-gRNA vectors are deposited at the China National Rice Research Institute in Hangzhou, China, and have been disclosed in the literature titled “Upgrading the genome of an elite japonica rice variety Kongyu 131 for lodging resistance improvement”. The original article link is as follows: onlinelibrary.wiley.com/doi/10.1111/pbi.1396.

In an embodiments of the present disclosure, the editing includes knockout.

The present disclosure does not particularly limit the method of construction of the recombinant vector, which is conventional in the art.

The present disclosure also provides a method for increasing arsenic tolerance in rice by utilizing a CRISPR/Cas9 system, including the step of
introducing the recombinant vector as described in the above scheme into a recipient rice to edit OsHIPP36 gene.

The present disclosure also provides a mutant of OsHIPP36 gene related to arsenic tolerance in rice, where the mutant of the OsHIPP36 gene includes a oshipp36-1 mutant and/or a oshipp36-3 mutant; the oshipp36-1 mutant has a deletion of an A base at position 205 bp within the OsHIPP36 gene fragment having the nucleotide sequence of SEQ ID NO: 2; the oshipp36-3 mutant has a G base inserted between positions 205 bp and 206 bp within the OsHIPP36 gene fragment having the nucleotide sequence of SEQ ID NO: 2.

The present disclosure also provides the use of the mutant of the OsHIPP36 gene as described in the above scheme in the regulation of arsenic tolerance in rice.

In an embodiments of the present disclosure, the use of the oshipp36 mutant described in the above scheme in regulating arsenic tolerance in rice includes the use of an overexpressed oshipp36 mutant in increasing arsenic sensitivity in rice.

The present disclosure also provides a set of primers for identifying the mutant of the OsHIPP36 gene as described in the above scheme, including an upstream primer having the nucleotide sequence of SEQ ID NO: 6 and a downstream primer having the nucleotide sequence of SEQ ID NO: 7, where the upstream primer having the nucleotide sequence of SEQ ID NO: 6 is specifically as follows: CTGCCGCCTCTCGTTTCTTTCT; the downstream primer having the nucleotide sequence of SEQ ID NO: 7 is specifically as follows:

GCGTTGGTAGGGTTGCCTCTTG.

To further illustrate the present disclosure, the use of the OsHIPP36 gene in regulating arsenic tolerance in rice provided by the present disclosure is described in detail below with reference to the accompanying drawings and examples, which should not be construed as limiting the scope of protection of the present disclosure.

### Example 1

### Sequence Analysis of OsHIPP36 Gene

To obtain a full-length CDS of the OsHIPP36 gene, total RNAs were extracted from the wild-type rice cultivar Nipponbare and reverse-transcribed into cDNAs. The CDS sequence of the OsHIPP36 gene was amplified by using the total cDNAs of Nipponbare as a template. After sequencing and analyzing the PCR product, the CDS of the OsHIPP36 gene was obtained, as set forth in SEQ ID NO: 2.

The amplification primer sequences were as follows:

upstream primer:
(SEQ ID NO: 8)
5′-ATGGGGGATGAGAAGGCTGC-3′;
downstream primer:
(SEQ ID NO: 9)
5′-TCACATCACCGAGCAGGCGT-3′

The PCR amplification system is shown in Table 1.

TABLE 1
PCR amplification system for amplifying
CDS sequence of OsHIPP53 Gene

| Reagents | 50 μL system | |
|---|---|---|
| 2 × PCR buffer for KOD FX | 25 | μL |
| 2 mM dNTPs | 10 | μL |
| Upstream primer | 1.5 | μL |
| Downstream primer | 1.5 | μL |
| KODFX enzyme | 1 | μL |
| Template cDNA | 2 | μL |
| ddH₂O | 9 | μL |

The PCR amplification reaction procedure was as follows: pre-denaturation at 94° C. for 2 min; denaturation at 98° C. for 10 s, annealing at 58° C. for 30 s, extension at 68° C. for 40 s, with a total of 29 cycles; complete extension at 68° C. for 10 min.

Through analysis of the coding region of the OsHIPP36 gene, it is determined that the OsHIPP36 gene contains 4 exons and 3 introns. The OsHIPP36 gene's cDNA sequence consists of 1047 nucleotides which encode 348 amino acids in total.

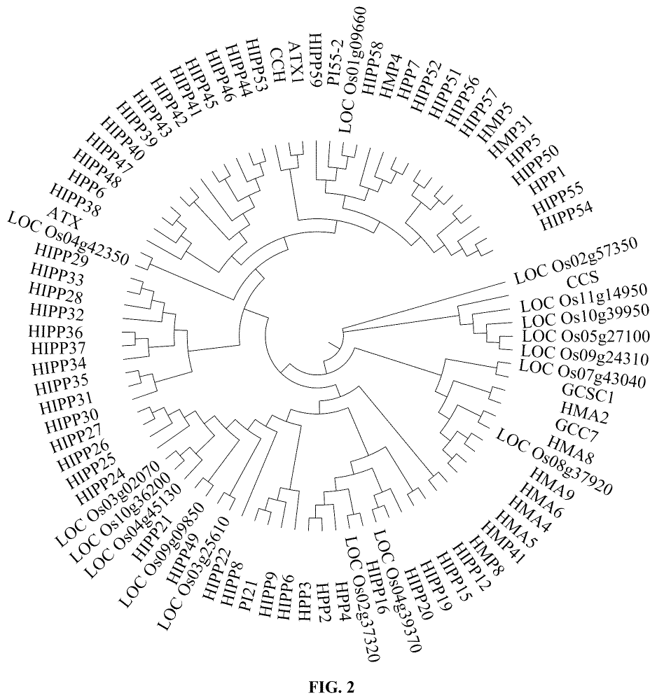

Phylogenetic analysis of the OsHIPP36 gene was performed using TBtools software. The results show that the OsHIPP36 gene belongs to the HIPP family and contains two HMA domains (FIG. 2).

### Example 2

Obtaining the oshipp36 mutant lines, i.e., oshipp36-1 and oshipp36-3 mutants, and identification of homozygous mutant lines.

Based on the coding sequence of the OsHIPP36 gene and the target design principles of CRISPR/Cas9, the target sgRNA sequence for constructing mutant materials was designed as follows:

target sgRNA:
(SEQ ID NO: 3)
5′-CGCGCTCAAGGCGAGGATCG-3′;

sgRNA-S, sgRNA-A and ddH₂O were mixed at a volume ratio, of 5:5:15. The mixture was incubated in a metal bath at 95° C. for 3 min, then the mixture was allowed to cool naturally at room temperature for 10 min to obtain the oligo dimer for later use; the SK-gRNA vector was digested by using the AarI restriction enzyme under conditions of 50° C. water bath for 1 hour, followed by gel extraction.

The oligo dimer and the enzyme-digested SK-gRNA product obtained above were ligated using T4 ligase. The volume ratio of the digested SK-gRNA product, oligo dimer, T4 ligase, and T4 Buffer was 1:7:1:1. After mixing, the reaction was incubated at room temperature for 4 h, then transformed into DH5a competent cells. Positive clones were screened using ampicillin-containing medium and identified by colony PCR. Sequencing confirmed the successful construction of the intermediate recombinant vector.

The intermediate recombinant vector obtained above was digested with KpnI and BglII restriction enzymes under conditions of 50° C. water bath for 1 h; the pC1300-Cas9 vector was digested with KpnI and BamHI restriction enzymes under conditions of 50° C. water bath for 1 h; Vector ligation was performed using T4 ligase. The volume ratio of the digested SK-gRNA intermediate recombinant vector product: digested pC1300-Cas9 vector product: T4 ligase: T4 Buffer was set at 4:4:1:1. After mixing, the reaction was incubated at room temperature for 4 h to obtain the recombinant vector.

The constructed recombinant vector was transformed into DH5a competent cells, and positive clones were screened using kanamycin-containing medium, resulting in positive clone cultures; recombinant plasmid extraction was then performed on the obtained positive clone cultures. Subsequent transgenic work was entrusted to Wuhan Bioyear Biotechnology Co., Ltd. for completion.

The obtained transgenic plants were identified by extracting DNA from both wild-type Nipponbare and the plant samples to be tested. PCR amplification was performed using the mutant identification primers described below, followed by Sanger sequencing. The obtained sequencing results were compared with those from Nipponbare PCR amplification to determine whether the OsHIPP36 gene mutation was successful.

The identification primer sequences are as follows:

OsHIPP36-F:
(SEQ ID NO: 6)
CTGCCGCCTCTCGTTTCTTTCT
OsHIPP36-R:
(SEQ ID NO: 7)
GCGTTGGTAGGGTTGCCTCTTG

See Table 2 for PCR amplification systems.

TABLE 2
PCR amplification system for identification primers.

| Reagents | 20 μL system | |
|---|---|---|
| 2 × Rapid Taq Master Mix | 10 | μL |
| OsHIPP53-F | 1 | μL |
| OsHIPP53-R | 1 | μL |
| Sample DNA | 1 | μL |
| ddH₂O | 7 | μL |

The PCR amplification reaction procedure was as follows: pre-denaturation at 95° C. for 3 min; denaturation at 95° C. for 15 s, annealing at 60° C. for 15 s, extension at 72° C. for 15 s, repeated for 32 cycles; complete extension at 72° C. for 5 min.

Two homozygous mutant lines were obtained after analysis of the sequencing results of the transgenic plants, named as oshipp36-1 and oshipp36-3 respectively. Sequencing results indicate that the oshipp36-1 mutant has a single-base deletion, specifically the deletion of an A base at position 205 bp within the OsHIPP36 gene coding nucleotide sequence; the oshipp36-3 mutant has a single-base insertion, with a G base inserted between positions 205 bp and 206 bp within the OsHIPP36 gene coding nucleotide sequence.

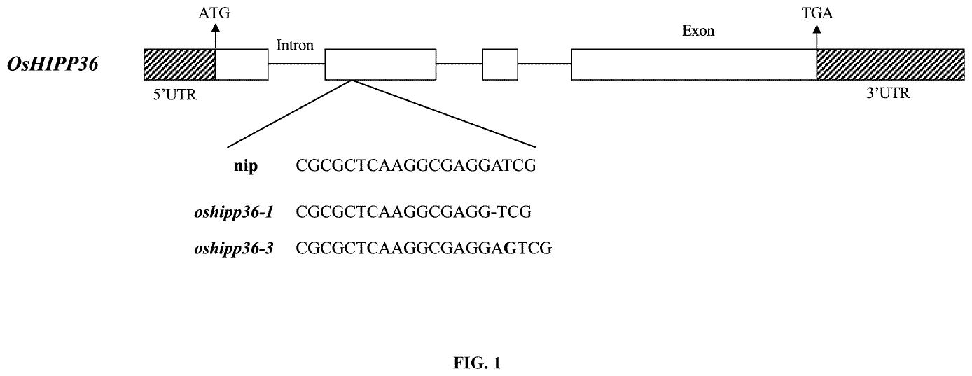

The identification results of the mutation sites in the OsHIPP36 transgenic plants are shown in FIG. 1.

### Example 3

Verification of the tolerance of oshipp36 mutants to As(III) treatment.

To determine the regulatory effect of the OsHIPP36 gene on arsenic tolerance in rice, using wild-type Nipponbare, oshipp36-1, and oshipp36-3 as experimental materials, As(III) treatment was simultaneously applied to observe their respective phenotypic changes. Rice seedlings were grown to 7 days of age, and seedlings of consistent growth were picked and transferred to Kimura B rice nutrient solutions with or without 10 μM NaAsO₂ for further cultivation. After 7 days of culture, the rice plants were subjected to phenotypic data collection and determination, including plant height, root length and biomass.

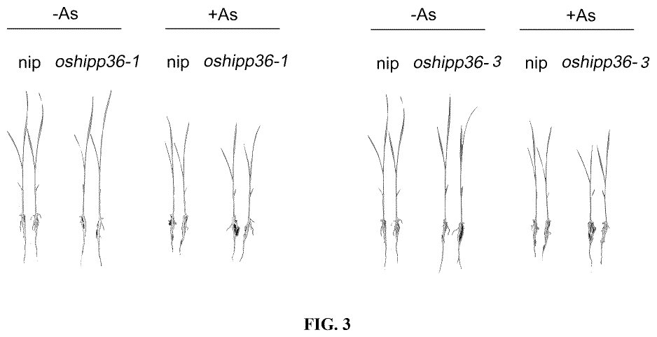

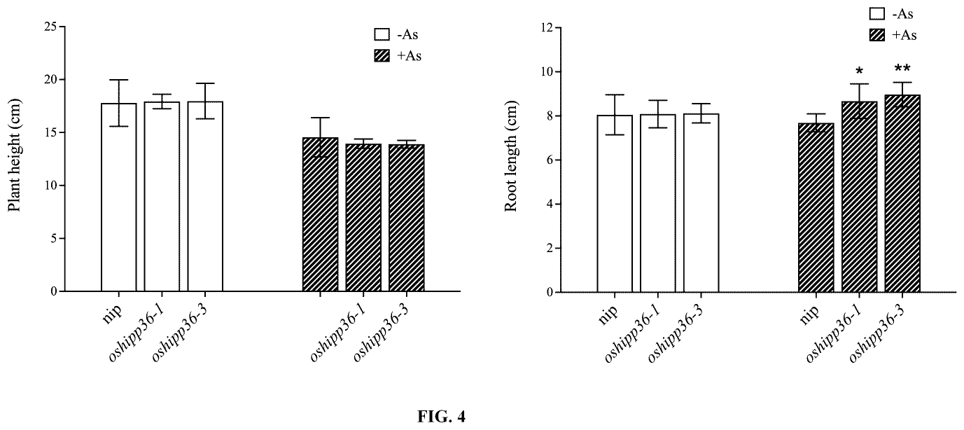

Under treatment conditions free of arsenic, oshipp36-1 and oshipp36-3 were essentially identical to wild-type Nipponbare in terms of growth; Under the 10 μM As(III) treatment condition, the growth of oshipp36-1 and oshipp36-3 was significantly superior to that of Nipponbare plants, as shown in FIG. 3. The plant height and root length of wild-type Nipponbare and mutant plants were measured under both normal and arsenic treatment conditions. Under normal conditions, there was no difference in plant height or root length between the mutants and Nipponbare. However, under arsenic treatment, the root length of the mutant plants was significantly longer than that of Nipponbare, as shown in FIG. 4. Based on the above results, it can be concluded that the mutant plants oshipp36-1 and oshipp36-3 exhibit higher tolerance to As(III), and that the OsHIPP36 gene plays a regulatory role in arsenic tolerance in rice.

In summary, the present disclosure employs genetic engineering techniques to mutate the OsHIPP36 gene. Through screening, the mutant lines oshipp36-1 and oshipp36-3 of the OsHIPP36 gene were obtained. Experimental validation confirmed that under arsenic treatment conditions, all phenotypic parameters of these mutant lines were superior to those of the wild-type Nipponbare, indicating that the mutant lines exhibited enhanced arsenic tolerance. The present disclosure experimentally demonstrated for the first time that the OsHIPP36 gene increases rice sensitivity to arsenic. In light of its application under arsenic stress, the OsHIPP36 gene was considered to have potential value for enhancing rice tolerance to arsenic stress. Furthermore, the present disclosure also established a solid theoretical and applied foundation for utilizing the OsHIPP36 gene in breeding arsenic-tolerant rice varieties.

Although the examples described above have provided a detailed description of the present disclosure, they are only some, rather than all, of the examples of the present disclosure. All other examples that may be obtained according to the examples of the present disclosure without involving any inventive effort shall fall within the scope of protection of the present disclosure.

1. (canceled)

2. (canceled)

3. (canceled)

4. (canceled)

5. (canceled)

6. (canceled)

7. A method for increasing arsenic tolerance in rice using a CRISPR/Cas9 system, wherein the method comprises the step of
introducing a recombinant vector into a recipient rice to edit OsHIPP36 gene;
wherein a CDS sequence of the OsHIPP36 gene has the nucleotide sequence of SEQ ID NO: 2;
the editing comprises deleting an A base at position 205 bp within the OsHIPP36 gene fragment having the nucleotide sequence of SEQ ID NO: 2, or inserting a G base between positions 205 bp and 206 bp within the OsHIPP36 gene fragment having the nucleotide sequence of SEQ ID NO: 2;
the recombinant vector comprises a sgRNA;
the sgRNA comprises a sgRNA-S having the nucleotide sequence of SEQ ID NO: 4 and a sgRNA-A having the nucleotide sequence of SEQ ID NO: 5.

8. (canceled)

9. (canceled)

10. (canceled)
